(19) United States
(12) Patent Application Publication
(10) Pub. No.: US 2008/0161750 A1
(43) Pub. Date: Jul. 3, 2008

(54) Oral Fluid Delivery or Sampling Device

(76) Inventor: Anne-Marie Pontis, Saint Remy Les Chevreuse (FR)

Correspondence Address: YOUNG & THOMPSON, 209 Madison Street, Suite 500, ALEXANDRIA, VA, 22314, US

(21) Appl. No.: 10/591,704

(22) PCT Filed: Mar. 8, 2005
(86) PCT No.: PCT/FR2005/000546

§ 371 Date: Sep. 6, 2006

(30) Foreign Application Priority Data
Mar. 10, 2004 (FR) 0402471

(51) Int. Cl. A61M 5/14

(52) U.S. Cl. 604/27

(57) ABSTRACT

The invention relates to an oral fluid delivery device operative with little or no deliberate intervention on the part of the user, particularly a weakened or unconscious user, or a user unable to use his or her hands. The invention is also useful for collecting an oral fluid sample, particularly for drainage or analysis purposes. Accordingly, the invention proposes an oral insert (2) designed to remain inside the mouth (10) of a user (1) for a prolonged period and provided with at least one port (20) for delivering or collecting a fluid. When the mouth is at rest, the oral insert takes up a large enough space in the oral cavity to withstand a force deliberately exerted in the direction of withdrawal (E) until the mouth is opened voluntarily.

The invention relates to a device enabling the oral administration of a fluid, which can be used with little or no voluntary action on the part of the user, in particular by a user who is weak or unconscious or who cannot use their hands. The invention may also enable an oral fluid sampling to be carried out, in particular for the purposes of drainage or analysis.

Different situations can exist, where a human or animal user may need to receive a fluid in a regular or repeated manner, without being capable of drinking by themselves or using a vessel in a normal way. Depending on the situation, the fluids to be administered may be simply water, a drink, or a liquid constituting or containing a chemical or medicated ingredient. These fluids may also have a certain viscosity, such as for example food blends. It may also be one or more gases, for example oxygen or an anaesthetic gas.

Apparatuses intended are known for users who cannot use their hands, for example sportspeople, workers with particularly cumbersome loads, or people wearing space suits or thermal protection suits. An apparatus intended for this type of use is common in sports shops, and described for example by the U.S. Pat. No. 5,911,406. An apparatus comprising a flexible backpack reservoir is connected via a flexible tube to a nozzle held with the teeth or the lips, and activated by sucking or by tongue pressure. However, this type of device requires a conscious and permanent action to keep the nozzle in the mouth and to use it, which can be a nuisance for example during strenuous activities or activities which call for concentration.

Other users, for example people who are ill or bedridden, can be incapable of drinking in the normal way because they are too weak physically or mentally. Certain medical situations also require a particularly frequent or eve n continuous hydration, optionally with the administration of a medical treatment. Thus, EP-A-0 349 261 describes a device comprising a valve supplying a nozzle in the form of a tube or a nipple, passing between the teeth, held by the teeth or by a flexible diaphragm which slides between the lips and the teeth or the gums. Feeding is then controlled by suction on the nozzle, or by action of the tongue on an opening provided in the nozzle. However, this type of device has drawbacks, for example the risk of the nozzle being damaged by the teeth. It can also be a source of discomfort due to the diaphragm inserted under the lips, which interferes for example when speaking, or risks irritating the mucosa on its contours. In addition, the requirement for even a small action means that this type of device is not very well suited to users who are not conscious, for example, sleeping or unconscious. Similarly, the use requires a certain capacity for understanding which is absent in the case of major mental disorders, or in a veterinary application.

In the case of a user who is occupied or moving, such as a sportsperson or in certain work situations, such a device can also be something of a nuisance. For example, it can interfere with breathing or speaking.

Moreover, numerous devices known in this field use dilution or flow-rate control devices such as spring-loaded valves, which are sometimes rather complex.

Until now, responses to these sorts of requirements consisted of setting up an intravenous perfusion using a needle, which is quite an invasive and delicate procedure, as well as being a source of risks and discomfort.

In addition, certain situations may involve the use of a device enabling a sampling of oral fluid. It may for example be a matter of carrying out drainage, such as during dental surgery or stopping an excess of saliva or post-operative seepage. It may also be a matter of carrying out continuous sampling for an analysis monitoring certain physiological factors. It may also be a matter of extracting a gas, for example an expiration gas in order to provide assistance or regular monitoring analysis.

A purpose of the invention is to propose a device which is easier or more comfortable for the user to hold in their mouth, in particular, without using hands or during strenuous activity.

Another purpose is to propose a device which may be applied to a user who is not able to voluntarily participate in its use.

Another purpose is to limit the interference for the user, in particular for breathing, speaking, or for various movements.

This objective is achieved by a device for the oral administration or sampling of a substance, comprising at least one oral insert (2) intended to be held in a continuous manner in the mouth (10) of a user (1) and comprising transfer means (20) for supplying thereto or sampling therefrom at least one substance (F), characterized in that, when the mouth is at rest, the oral insert occupies a sufficient volume in the oral cavity to exert a resistance to a determined extraction force (E) as long as the mouth is not voluntarily opened. The device may in particular comprise an extra-gingival part intended to be inserted into a part of the oral cavity (10) situated inside one cheek (15) and outside one maxilla.

Such a positioning and retention method make it possible to envisage a design which significantly unblocks the mouth opening, which enables a better breathing airflow for the effort and interferes less with speaking.

These transfer means may be one or more orifices, or a structure which enables the fluid F to pass through. These transfer means may also deliver or sample the substance F by diffusion or osmosis.

According to one feature, the oral insert comprises at least one part which can be deformed under the pressure of the walls of the mouth.

According to one feature, the oral insert comprises at least one part which can decrease in volume under the pressure of the walls of the oral cavity.

The insert can thus adjust to the internal conformation of the user's mouth, or assume a shape complementary to its internal surfaces, improving the comfort and holding.

According to one feature, the oral insert comprises at least one part inserted inside the maxillae and which has on its front side a bulge which bears on the internal surface of the teeth or the gum of at least one maxilla, thus exerting a resistance to an extraction force.

Thus, whatever its shape initially or after insertion and/or deformation, a part of the oral insert may have a shape which is approximately complementary to the shape of the palate, and can be held easily and naturally in a continuous manner, pushed by the tongue against the palate.

In the same way, a part of the oral insert may comprise at least one so-called extra-gingival part, inserted into a part of the oral cavity situated inside one cheek and outside one maxilla. This extra-gingival part may also have, once in place, a shape approximately complementary to an external surface of at least one tooth or at least one gum with which it is in contact and bearing on this surface in order to exert a resistance to an extraction force.

Holding may also be improved by an oral insert which comprises at least one part having, once in place, a surface with a shape complementary to a surface of the oral cavity with which it is in contact and cooperating with this surface in order to exert, by a suction effect, a resistance to an extraction force.

Another purpose is to simplify the flow-rate or dilution control systems.

To this end, the preceding characteristics may also be combined with an oral insert comprising several orifices between which there is distributed a flow of fluid transferred between the oral cavity and the inside of the oral insert.

For the same purpose, at least one part of the oral insert may be constituted by a porous material which distributes the delivered or sampled flow of fluid over a large part of its external surface.

According to one feature, the insert comprises a storage chamber communicating with the oral cavity in order to deliver thereto or sample therefrom at least one fluid.

According to one feature, the oral insert delivers a product in solid form or with high viscosity and interacting with a second fluid inside the oral insert, the product having after this interaction a sufficiently reduced viscosity to enable or to increase its flow rate passing through at least one orifice communicating with the oral cavity.

According to one feature, these characteristics may be combined in a device where the oral insert is autonomous, or even without an element outside the mouth, which enables the user greater freedom as well as precise knowledge of the maximum quantity of fluid delivered.

According to another feature, these characteristics may also be combined in a device where the oral insert enables the oral cavity to communicate with at least one conduit outside the user and conveying at least one administered or sampled fluid into this oral cavity.

This conduit may then serve to supply the insert with fluid or to aspirate the fluid from it, by all known methods.

Other characteristics and advantages of the invention will become apparent from the detailed description of an embodiment which is in no way limitative, and from the attached drawings in which:

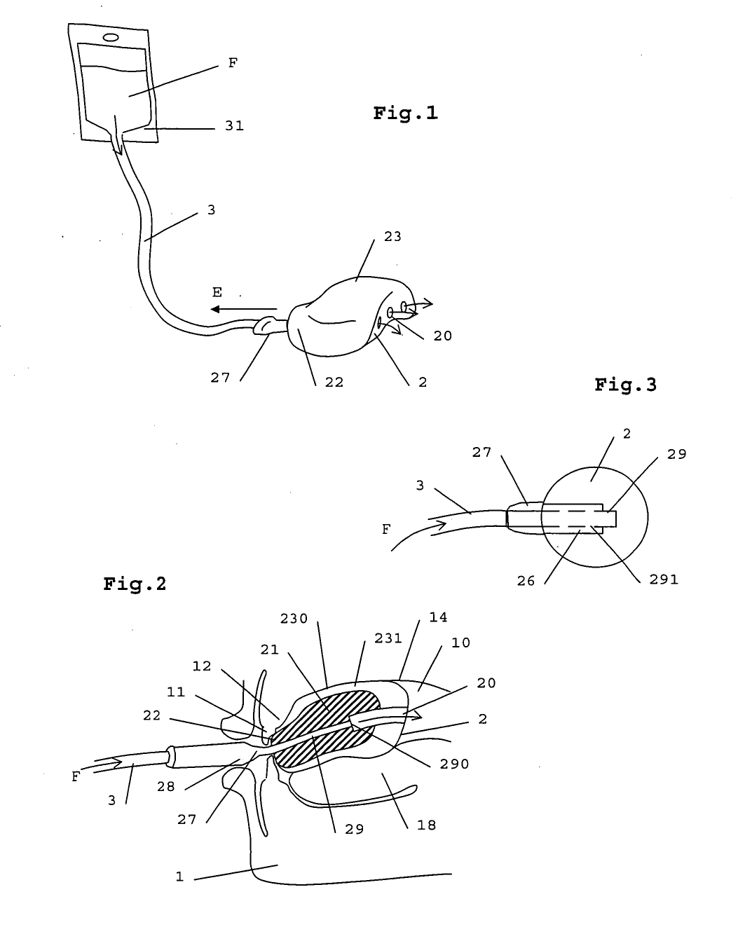

FIG. 1 illustrates an embodiment of the invention comprising an oral insert to be placed against the palate and supplied with fluid by a raised bag;

FIG. 2 is a partial sagittal cross-section of an embodiment of the invention with the oral insert in place under the palate and connected via an external conduit;

FIG. 3 is a partial top view in cross-section of an embodiment of the invention with an oral insert in the form of a rigid porous rounded pebble;

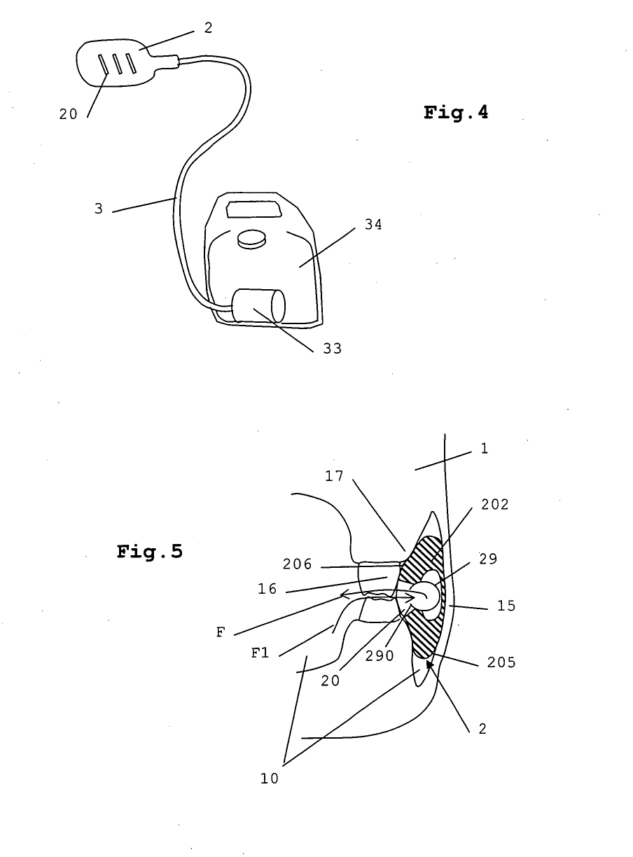

FIG. 4 illustrates an embodiment of the invention comprising an oral insert to be placed inside the cheek and supplied with fluid by a reservoir bag with a pressurizing pump;

FIG. 5 represents a partial cross-section perpendicular to the sagittal plane of an oral insert according to the invention, to be placed inside the cheek and comprising a flexible part which conforms to the space between cheek and maxilla;

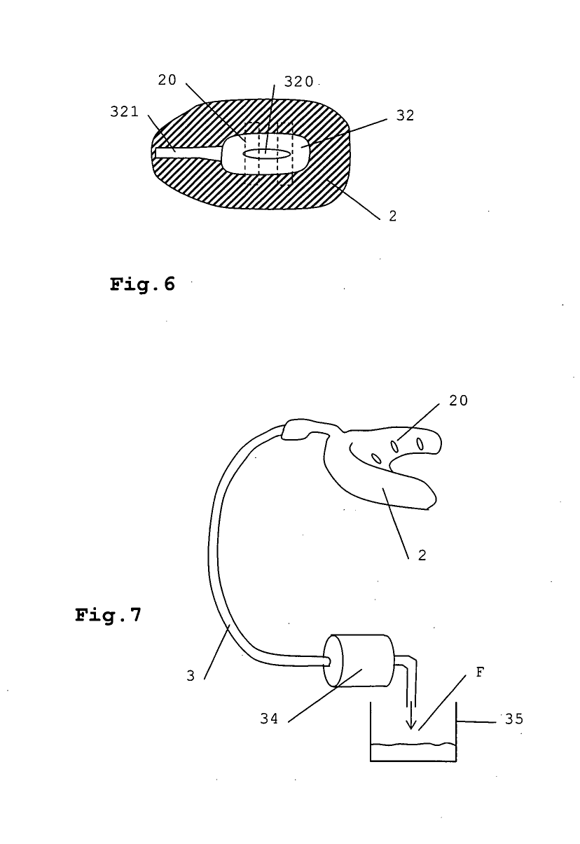

FIG. 6 illustrates an embodiment of the invention where the oral insert comprises an internal storage chamber and may be used without external connections;

FIG. 7 illustrates an embodiment of the invention comprising an oral insert to be placed under the tongue and connected to a sampling pump.

In an embodiment of the invention illustrated in FIGS. 1 and 2, an oral insert 2 in the general shape of a pad is provided with internal conduits opening into one or more orifices 20 constituting transfer means between the inside of the insert and the oral cavity. These orifices communicate with a flexible tube forming a conduit 3 which is connected to the oral insert by a nozzle 27. This nozzle has a flattened part limiting the opening of the jaws which is required for the conduit to pass through. This flattened part forms a groove flanked by an external bulge 28 enabling the nozzle 27 to be easily held between the teeth 11.

A fluid F is conveyed to the oral insert 2 via the conduit 3 by known means, for example from a reservoir or from a raised bag 31 similar to those used for perfusions. The fluid may also be pressurized by other known means, or aspirated by the user 1 through the orifices 20 of the oral insert 2 and the conduit 3.

In this embodiment, the oral insert 2 has on its upper part a dome 23 which has a shape approximately complementary to that of the hard palate 14 and is placed against the latter. Once in place in the oral cavity 10 of the user 1, between the tongue 18 and the palate 14, the oral insert has a sufficiently large volume to be retained by the front teeth 11 or their gum 12 when a certain extraction force E is applied to it, for example, by slight movements of the head, or is applied to the conduit 3, or simply by the weight of this conduit. This resistance is created in particular by the volume of the insert, which requires a voluntary action of the user or a person outside in order to pass between the teeth or the jaws. In fact, when they are at rest, the muscles of the jaw tend to exert a slight closing effort which exists even when the person is not thinking about it or is not conscious, for example when sleeping.

This resistance may be improved by a bulge 22 situated on the peripheral part of the oral insert. In particular, the front surface of this bulge 22 may have a shape complementary to that of the teeth 11 and/or the upper and/or lower gums 12 against which it bears.

The external shape of the dome 23 of the insert, or of its bulge 22, or of any other part in contact with the walls of the oral cavity 10 of the user 1 may be produced in a shape approximately complementary to that of the corresponding wall of the oral cavity, or may be obtained by moulding to this wall.

As shown in FIG. 2, the oral insert also comprises parts which are deformable or can be crushed, which enables to obtain such a complementary and comfortable contact with the walls of the oral cavity 10. The nozzle 27 extends inside the oral cavity at one internal end 29 provided with orifices 290, communicating with the external orifices 20 opening into the oral cavity 10. Around this end 29, the oral insert comprises a part forming a core 21 determining the general shape of the insert. Advantageously, this core 21 is of a material and/or has a structure which is flexible and deformable which enables a certain adaptation to the internal conformation of the oral cavity 10. Around this core 21, the oral insert 2 comprises a part forming a partial or total covering 231. This covering 231 is of a compressible material and/or structure, which enables a comfortable contact of its external surface 230 with the wall of the oral cavity, in particular the hard palate 14.

The complementarity of the contact between the wall of the oral cavity and/or the teeth 11 or gums 12 also enables the oral insert 2 to be retained by a suction effect at these contact points, in particular, due to the presence of the fluid F or of saliva.

The movement of the fluid F between the conduit 3 and the oral cavity 10 may take place through the orifice or orifices 290 of the nozzle communicating with orifices 20 of the external rear surface of the oral insert. This movement may also take place through a porosity of all or part of the materials or structures constituting this insert. For example, the core 21 and the covering 231 may be produced from an open-bubble foam, which ensures the communication of the fluid while distributing or diffusing its flow over a large part of the external surface of the insert.

In an embodiment illustrated in FIG. 3, the oral insert has a rounded shape without sharp edges, for example ovoidal or in the shape of a pebble. This pebble may be made of a material and/or have a structure which is porous and only slightly or not at all deformable. This insert surrounds the internal end 29 of the nozzle 27, creating around it a cavity 26 communicating with the orifices 290 of the nozzle and enabling a diffusion of the fluid F in the thickness of the insert.

In an embodiment illustrated in FIGS. 4 and 5, an oral insert 2 has on its external surface one or more orifices 20 with an oblong shape or in grooves. These orifices communicate via the conduit 3 with a pump 33 attached to a reservoir bag 34 supplying this pump.

Conversely, the pump 33 may also, selectively or exclusively, aspirate fluid originating from the mouth via the conduit 3 and deliver it to the reservoir bag 34.

In the embodiment shown in FIG. 5, the oral insert 2 is positioned on the side of the mouth, inside the cheek 15 and outside the maxillae bearing the lateral teeth 16 and their gums 17. The oral insert 2 then comprises a deformable part 202 surrounding the end 29 of the nozzle 27, and enabling communication between the orifices 20 of the insert and the orifices 290 of the end 29 of the nozzle 27. Under the pressure of the cheek 15, this deformable part 202 adjusts such that it is approximately complementary to the shape of the cheek, as well as the lateral teeth 16 and their upper and/or lower gums 17. This complementarity creates irregularities of shape which interact with the shape of the teeth and combines with friction to enable the oral insert to be retained in its position.

Moreover, the complementarity of the deformable part 202 with the internal surface 205 of the cheek 15 and with the surface 206 of the teeth and the gums on which it bears enables it to cooperate with these surfaces in a suction effect, thus exerting an additional resistance to an extraction force E.

In a manner which is not shown, the part 202 may even have a rib intended to be inserted between the top teeth and the bottom teeth.

In an embodiment which may be applied with several of the configurations described here, the fluid F may be presented in a form with a high viscosity, for example a gel, which allows only little or no passage through the orifices 290, 20 inside the oral cavity 10. This fluid F may then be diluted or modified by interaction with a fluid F1 present in the oral cavity, for example saliva or a fluid supplied for other reasons or a gas which is inhaled or exhaled, and penetrating into the oral insert. After interaction with this second fluid F1, the fluid F to be administered can then more easily exit the oral insert and pass into the oral cavity 10 itself. In an embodiment illustrated in FIG. 6, an oral insert 2 comprises an internal storage chamber 32, which stores the fluid F to be administered or sampled. This chamber comprises one or more chamber orifices 320 which may communicate with the orifices 20 of the oral insert opening into the oral cavity. This internal storage chamber 32 may be temporarily connected to an external conduit by a connection opening 321 which can be blocked or stopped up. This internal storage chamber 32 may also be filled once for all, in order to produce a disposable administration device. During its storage in the chamber 32, the fluid F to be administered may also be presented in a non-fluid storage form. This form may for example be a gel or a solid which can be melted, diluted or disintegrated under the action of a fluid F1 originating from the oral cavity when the insert is placed therein. The storage chamber 32 may also contain another substance, called a precursor, different from the fluid F, the interaction of which with the fluid F1 produces the fluid F to be administered.

This internal storage reservoir may also be in the form of a material or a structure containing the fluid F or its precursor, for example by impregnation. The oral insert itself, as a whole or in part, may constitute such a reservoir in the form of a material or a structure containing and delivering or diffusing the fluid F or its precursor.

In the embodiment illustrated in FIG. 7, an oral insert 2 is connected via the conduit 3 to suction means of a known type, for example a vacuum pump 34 or a venturi. This pump aspirates the fluid F out of the oral cavity through the orifices 20 of the oral insert, and conveys it for removal or storage, for example to a receptacle 35. In this embodiment, the oral insert 2 has a crescent shape approximately complementary to the lower part of the inside of the oral cavity of the user, or capable of being inserted therein. Once placed under the tongue, this insert has a certain volume which enables it to resist a certain extraction force as long as the mouth is not voluntarily opened, for example by lifting the tongue or through intervention by a carer.

For example it is possible to thus produce a device for the extraction of saliva during dental surgery, which has better stability than a simple curved tube placed on the teeth of the lower jaw which is often used in this field.

The different combinations of characteristics described here may of course be used to realize the invention in order to carry out both sampling and administration of fluid, or a combination of these two functions.

In a general way, each of the embodiments of the oral insert combines with any one of the embodiments of the external device with a pump or reservoir.

Of course, the invention is not limited to the examples which have just been described and numerous variations may be applied to these examples without exceeding the scope of the invention.

1-12. (canceled)

13. An oral device for administration or sampling of a substance, comprising at least one oral insert (2) intended to be kept in a continuous manner in the mouth (10) of a user (1) and comprising transfer means (20) for delivering thereto or sampling therefrom at least one fluid (F), said oral insert having a shape such that it can be housed entirely in a so-called lateral region of the oral cavity (10) situated outside a maxilla and inside a cheek (15), the interaction of the insert with the walls (15, 17) of said lateral region then producing a conjugation of shapes (205 with 15, 206 with 16) cooperating with each other so as to exert a resistance to extraction (E).

14. The device according to claim 13, wherein the oral insert (2) benefits of a holding due to the pressure of the cheek (15).

15. The device according to claim 13, wherein the oral insert (2) comprises at least one part (21) which can be deformed under the pressure of the walls of the oral cavity (14, 15).

16. The device according to claim 13, wherein the oral insert (2) comprises at least one part (231, 202) which can decrease in volume under the pressure of the walls of the oral cavity (14, 15).

17. The device according to claim 13, wherein the oral insert (2) is arranged in such a way that, once it is in place, it has a shape complementary to an external surface of at least one tooth (16) or at least one gum (17) with which it is in contact and bearing on this surface so as to exert a resistance to an extraction force (E).

18. The device according to claim 13, wherein the oral insert (2) comprises at least one part arranged in such a way that, once it is in place, it has a surface (230, 205, 206) with a shape approximately complementary to a surface of the oral cavity (10) with which it is in contact and cooperating with this surface (14, 15, 16, 17) so as to exert by a suction effect a resistance to an extraction force (E).

19. The device according to claim 13, wherein the oral insert (2) comprises several orifices (20) between which there is distributed a flow of fluid (F) transferred between the oral cavity (10) and the inside of the oral insert (29, 32).

20. The device according to claim 13, wherein at least one part of the oral insert (2) is constituted by a porous material through which the flow of delivered or sampled fluid (F) flows in a distributed manner.

21. The device according to claim 13, wherein the oral insert (2) comprises a storage chamber (32) communicating with the oral cavity (10) in order to deliver thereto or sample therefrom at least one fluid (F).

22. The device according to claim 13, wherein the oral insert (2) enables the oral cavity (10) to communicate with at least one conduit (3) outside the user (1) and conveying at least one fluid (F) administered or sampled in this oral cavity.

23. The device according to claim 13, wherein it delivers a product (F) initially in solid form or with a high viscosity and interacting with a second fluid (F1) inside the oral insert (2), the product (F) having after this interaction a sufficiently reduced viscosity to allow or to increase its flow rate passing through at least one orifice (20) communicating with the oral cavity (10).

24. A method for orally continuously or repeatedly administrate or sample a substance (F), said method comprising keeping in a user mouth (10) an oral insert (2) according to claim 13.
